(19) United States
(12) Patent Application Publication
(10) Pub. No.: US 2016/0367959 A1
(43) Pub. Date: Dec. 22, 2016

(54) Multiphase Compositions

(71) Applicant: Avon Products, Inc., Suffern, NY (US)

(72) Inventors: Saundra R. LAPRIORE, Shohola, PA (US); Andrew H. Pechko, Ridgewood, NJ (US); Mark S. Naser, Franklin, NJ (US)

(21) Appl. No.: 14/743,079

(22) Filed: Jun. 18, 2015

(51) Int. Cl. B01F 17/00; A61K 8/92; B65D 25/54; A61Q 19/00; B65D 25/38; A61K 8/06; A61K 8/39

(52) U.S. Cl. CPC B01F 17/0092; A61K 8/066; A61K 8/92; A61K 8/39; A61Q 19/00; B65D 25/38; B65D 25/54

(57) ABSTRACT

The invention relates generally to multi-phase compositions that comprises of three mutually immiscible liquids. One of the liquids comprises a triglyceride oil, another comprises a silicone oil, and a third liquid comprises a polyalkylene glycol. The invention also relates to packaged products, such as cosmetic or personal care products comprising a container comprising a transparent or translucent material to view a multi-phase composition contained therein and a closable aperture for dispensing the multi-phase composition.

### FIELD OF INVENTION

The invention relates generally to compositions comprising a plurality of immiscible phases which form visually distinct layers on standing. Such compositions find utility in cosmetic and personal care products.

### BACKGROUND

Multi-phase compositions for cosmetics or personal care are generally known. Examples include emulsions, in which at least one liquid is dispersed as colloidal droplets within another liquid. The most prevalent types of emulsions, water-in-oil and oil-in-water, are aqueous. Emulsions typically employ the use of emulsifiers to prevent coalescence of the colloidal droplets and consequent separation of the phases into discrete layers. In some applications, however, it may not be desirable to include substantial levels of emulsifiers.

It is also known to provide multi-phase compositions in the form of two or more liquids forming separate layers. Most multi-phase compositions take advantage of the well-known immiscibility of water and oils. For example, three layer systems have been developed that employ, in addition to water and oil phases, an alcohol or polyol phase (e.g., hexylene glycol, sorbitol, glycerin, etc.). Some multiphase compositions use agents such as salts, polysaccharides, ethoxylated fatty esters or ethers, to promote phase separation between a water and glycol phase. Others employ ethoxylated glyceryl fatty acid monoesters (e.g., PEG-glyceryl cocoate) as a separate phase, which is immiscible with water and oil phases. The usefulness or generality of these multiphase compositions is based on the inclusion of a water-phase and other components required to maintain phase separation. There is a need in the art for substantially anhydrous multi-phase compositions, and in particular multiphase compositions that do not rely on specific additives to induce phase separation.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, the present invention provides compositions comprising a plurality (e.g., three or more) of mutually immiscible liquid phases comprising, at least, a first phase having triglyceride oil (e.g., a vegetable oil) as the predominant or major component; a second phase having a silicone oil (e.g., a non-volatile, linear dimethicone fluid) as the predominant or major component; and a third phase having polyethylene glycol (PEG) as the predominant or major component. By “predominant component” is meant that the amount by weight of the indicated component is greater than the amount by weight of any other component in a given phase. By “major component” is meant that at least 50% of the weight of each phase is comprised of the indicated component. In various embodiments, each phase may be comprised of at least about 60%, at least about 65%, at least about 70%, at least about 75%, at least about 80%, at least about 85%, at least about 90%, at least about 95%, or about 100% by weight of the indicated component. In some embodiments, the first phase will comprise from about 50-100% by weight triglyceride oil, typically a plant-derived triglyceride oil such as a vegetable oil. In some embodiments, the plant-derived triglyceride oil will be comprised of at least about 25% (or at least about 30%, or at least, about 35%, or at least about 40%, or at least about 45%, or at least about 50%, or at least about 55%) by weight linoleic acid (based on the weight of the plant-derived triglyceride oil). In some embodiments, the plant-derived triglyceride oil will be comprised of at least about 10% (or at least about 15%, or at least about 20%, or at least about 25%, or at least about 30%) by weight oleic acid (based on the weight of the plant-derived triglyceride oil). In some embodiments, the plant-derived triglyceride oil will comprise linoleic and oleic acid in an aggregate amount of least about 50% (or at least about 60%, or at least about 70%, or at least about 80%, or at least about 90%) by weight, the remainder optionally comprising palmitic and stearic acids in an individual or aggregate amount from 1-25% (e.g., from 5-15%) by weight (based on the weight of the plant-derived triglyceride oil). In other embodiments, the first phase will comprise from about 50-100% (or 70-99% or 80-98% or 85-95%) by weight of a plant-derived triglyceride oil, such as sunflower oil. The first phase may contain additional oils that are miscible with the triglyceride oil, including, without limitation, additional triglyceride oils, vegetable oils, ester oils, vitamins (e.g., tocopherol and esters thereof, such as tocopheryl acetate), hydrocarbon oils, and fatty alcohols (e.g., C₁₂₋₁₈ fatty alcohols). The second phase will typically comprise a silicone oil, which is usually non-volatile. In some embodiments, the silicone oil comprises a linear silicone oil which may, for example, have a viscosity between about 5-100,000 cSt (at 25° C.). The linear silicone oil may comprise from about 50-100% (or 80-100% or 90-100% or 95-100%) by weight of the silicone oil phase. In one embodiment, the silicone oil phase is substantially free (e.g., less than 1% by weight) of cyclic silicones (e.g., D4, D5, etc.). The third phase comprises from about 50-100% (or 55-90% or 60-80% or 65-75%) by weight PEG. The PEG typically, but not necessarily, is liquid at room temperature and has a number average molecular weight between about 100-800 Daltons (e.g., between about 200-400 Daltons). In one embodiment, PEG will have a number average molecular weight of about 300 Daltons. The third phase may also comprise from about 1-50% (or about 15-45% or about 20-40% or about 25-35%) by weight of a lower alcohol, typically ethanol. In one implementation, the third phase comprises from 50-90% by weight of a liquid PEG having a number average molecular weight between about 100 and about 800 Daltons and about 10-40% by weight ethanol. The compositions may be substantially anhydrous, by which is meant that they comprise less than 5% by weight water, more typically less than 2.5% by weight water. In some embodiments, the compositions will comprise less than about 1% by weight water. The composition may be substantially free (less than 1%, or less than 0.5%, or less than 0.1% by weight) or free of salts (e.g., NaCl), ethoxylated glyceryl fatty acid monoesters (e.g., PEG-glyceryl cocoate), and/or polyols (e.g., hexylene glycol, butylene glycol, glycerin, etc.). In one embodiment, the compositions do not comprise an ethoxylated glyceryl fatty acid monoester (e.g., PEG-glyceryl cocoate) phase, by which is meant that no phase contains from 1-100% or from 5-100% or from 25-200% or from 50-100% or from 75-100% by weight ethoxylated glyceryl fatty acid monoesters, including PEG-glyceryl cocoate, and in particular PEG-7-glyceryl cocoate. The compositions of the invention are characterized in that the first, second and third phases are mutually immiscible, can appear to be a single phase composition after vigorous shaking or agitation, but thereafter will separate back into three visually distinct layers under quiescent conditions (e.g., standing undisturbed for 30 minutes). In some embodiments, the volume of the first, second and third phases will not change by more than ±5% (or less than about ±2.5% or ±1%) following ten cycles of shaking to create a single phase composition followed by standing for a time (e.g., 30 minutes) sufficient to permit the three layers to re-form. Typically, the specific gravity of the second phase (e.g., linear, non-volatile silicone oil) is greater than the first phase (e.g., vegetable oil) but less than the PEG containing third phase, such that said first phase forms a top layer, said second phase forms a middle layer, and said third phase forms a bottom layer, with the middle layer being in contact with the top and bottom layers. The top, middle, and bottom layers may have different refractive indices, and are typically visually distinct from one another. In some embodiments, the PEG containing layer will comprise an amount of colorant (e.g., at least about 0.0001% by weight) sufficient to impart a visible hue to the bottom layer. The compositions may include any conventional actives and excipients common in cosmetic and personal care products, including, without limitation, waxes, gelling agents, solvents, conditioning agents, emulsifiers, surfactants, emollients, humectants, film forming polymers, rheology modifiers, thickeners, stabilizers, sunscreens, botanicals, pH adjustors, chelators, preservatives, fragrances, and colorants, such additional ingredients being present, individually or in the aggregate, in an amount from about 0.001-50% (or about 0.001-25% or about 0.01-15%) by weight of the total compositions, or by weight of each phase. Such additional ingredients should not be included in amounts that cause any two of the phases to become miscible.

The composition may be a packaged product, such as a cosmetic or personal care product (e.g., a bath oil), comprising:

(a) a container, for example, one having a wall surrounding a chamber, wherein the wall or a portion thereof is composed of a transparent or translucent material such that contents contained within said chamber are visible from outside the container, said container having a closable aperture for dispensing the contents thereof; and

(b) a composition (typically, substantially anhydrous or anhydrous) disposed within said container (e.g., substantially filling said chamber), the composition comprising: (1) a first phase having triglyceride oil as the major component; (2) a second phase having a silicone oil as the major component; and (3) a third phase having polyethylene glycol (PEG) as the major component;

wherein said first, second and third phases are mutually immiscible and wherein the specific gravity of said second phase is greater than the first phase but less than the third phase, such that, on standing, said first phase forms a top layer, said second phase forms a middle layer, and said third phase forms a bottom layer, the middle layer being in contact with the top and bottom layers, and wherein said layers are visually distinct from one another; wherein said first phase, second phase, and third phases appear to form a single phase composition upon shaking, but separate back into distinct layers within minutes on standing. The first, second, and third phases may occupy approximately equal volumes in the container. For example, the first phase may occupy from 20-40%, the second phase may occupy from 20-40%, and the third phase may occupy from 20-40% of the chamber volume within the container. In one embodiment, the composition is capable of forming a single phase mixture on shaking, agitating, shearing, etc., such that when the product is dispensed from the aperture, approximately equal volumes of each phase are dispensed. At least one of the phases may comprise a colorant (such as a pigment, lake dye, pearl, etc.). At least two of the phases, or all three phases, may have a different hue.

These and other aspects of the present invention will become apparent to those skilled in the art after a reading of the following detailed description of the invention, including the appended claims.

### BRIEF DESCRIPTION OF DRAWINGS

FIG. 1 illustrates one embodiment of a packaged product 10 according to the invention. Packaged product 10 comprises a container 20 in the form of a bottle with transparent or translucent walls, and a cap 30 for closing the bottle. In this embodiment, the bottle is comprised of glass or a clear, transparent plastic (e.g., PET, HDPE, etc.). The cap 30 may be removable (e.g., screw-on) for dispensing the product through the mouth of the bottle. Alternatively, the cap may contain an aperture (not shown), through which product may be squeezed or poured, and lid or top for sealing the aperture when in a closed position. The packaged product contains a composition according to the invention, comprising three visibly distinct immiscible layers 40, 50, and 60. Top layer 40 comprises, as the major portion, sunflower oil, middle layer 50 comprises, as the major portion, non-volatile silicone oil (fluid), and lower layer 60 comprises, as the major portion, PEG having a number average molecular weight between about 100-800 Daltons and, as a minor portion, ethanol.

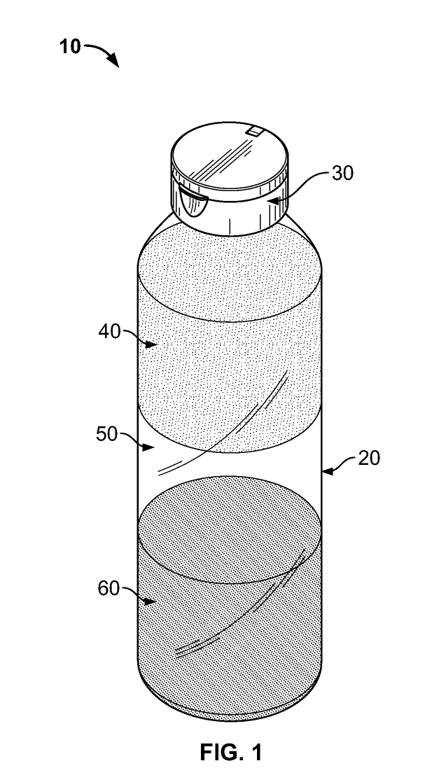

### DETAILED DESCRIPTION

All terms used herein are intended to have their ordinary meaning unless otherwise provided. All ingredient amounts provided herein are by weight percent of the total composition unless otherwise indicated. It will be understood that the aggregate weight or volume of all components of a compositions will not exceed 100%.

“Substantially anhydrous” as used herein means containing less than 5% water. In other embodiments, the vehicle and/or the entire cosmetic or personal care composition comprises less than 4%, less than 3%, less than 2%, less than 1%, less than 0.5%, or less than 0.1% by weight water. The term anhydrous as used herein means that no water is added to the composition and that only that amount of water absorbed from the atmosphere will be present in the composition. As used herein, the term “oil” is intended to refer to a water-insoluble material that is liquid at room temperature (i.e., 25° C.).

By “topically acceptable” is meant that an ingredient is generally regarded as safe and non-toxic for application to a human integument.

By “consists essentially of” is meant that additional ingredients are excluded in amounts that would affect the basic and novel features of the invention, including, for example, the ability of the three or more phases to form immiscible layers.

The compositions of the invention comprise at least three immiscible phases. Each of the phases is preferably visually distinct from at least one other phase. The first phase comprises a triglyceride oil. The triglyceride oil may constitute from about 10-100% or from about 25-100% or from about 50-100% or from about 75-100% of the first phase.

The triglyceride oil may comprise a single triglyceride oil or mixtures of triglycerides, oils and fats. The triglyceride oil will typically comprise glycerin esterified with one, two, or three fatty acids. The fatty acids will typically be C₆₋₂₆ or C₁₀₋₂₄ or C₁₂₋₂₂ fatty acids. The fatty acids may be saturated or unsaturated. Unsaturated fatty acids may comprise one, two, three, or more double bonds. In some embodiments, at least one fatty acid may be selected from unsaturated fatty acids, including linoleic acid (polyunsaturated omega-6), oleic acid (monounsaturated omega-9), and gamma-linolenic acid (GLA), or saturated fatty acids such as stearic acid and palmitic acid. In various embodiments, a particular fatty acid may comprise from 1-100% by weight of the fatty acid content of the triglyceride, including from 5-95%, or from 1-5%, 5-10%, 10-20%, 20-50%, 50-75%, or 75-95%.

In some embodiments, the triglyceride oil comprises a vegetable oil. Suitable vegetable oils, include, without limitation, Sunflower oil (e.g., sunflower seed oil), Coconut oil, Corn oil, Cottonseed oil, Olive oil, Palm oil, Peanut oil (Ground nut oil), Rapeseed oil, Canola oil, Safflower oil, Sesame oil, Soybean oil; Nut oils, including without limitation, Hazelnut oil, Almond oil, Beech nut oil, Brazil nut oil, Cashew oil, Hazelnut oil, Macadamia oil, Mongongo nut oil (or manketti oil), Pecan oil, Pine nut oil, Pistachio oil, Walnut oil; Citrus oils, such as Grapefruit seed oil, Lemon oil, and Orange oil, sea-buckthorn oil, Oils from melon and gourd seeds, Watermelon seed oil, Cucurbitaceae oils, Bitter gourd oil, Bottle gourd oil, Buffalo gourd oil, Butternut squash seed oil, Egusi seed oil, Pumpkin seed oil, Borage seed oil, blackcurrant seed oil, Evening primrose oil, Açai oil, Black seed oil, Blackcurrant seed oil, Borage seed oil, Flaxseed oil, Carob pod oil, Amaranth oil, Apricot oil, Apple seed oil, Argan oil, Avocado oil, Babassu oil, Ben oil, Borneo tallow nut oil, Cape chestnut oil, Carob pod oil (Algaroba oil), Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Manila oil, Meadowfoam seed oil, Mustard oil (pressed), Niger seed oil, Poppyseed oil, Nutmeg butter, Nutmeg oil, Okra seed oil, Papaya seed oil, Perilla seed oil, Persimmon seed oil, Pequi oil, Pili nut oil, Pomegranate seed oil, Pracaxi oil, Prune kernel oil, Quinoa oil, Ramtil oil, Rice bran oil, Royle oil, Shea butter, Sacha inchi oil, Sapote oil, Seje oil, Taramira oil, from the seeds of the arugula (Eruca sativa), Tea seed oil (Camellia oil), Thistle oil, Tigernut oil (or nut-sedge oil), Tomato seed oil, and Wheat germ oil, to name a few. In one embodiment, the triglyceride oil phase does not contain, or does not contain an appreciable amount of (in one embodiment more than 10% of) petroleum distillates and other hydrocarbons as described in the Mar. 3, 2011 CPSC “Guide to Child Resistant and Senior-Friendly Packages”, herein incorporated by reference in its entirety.

Vegetable oils may be present, individually or collectively, in an amount from about 5-100% by weight of the triglyceride phase, more commonly from about 10-100% or about 20-100% or about 50-100%, or about 75-100% or about 85-100% by weight of the triglyceride phase. In one embodiment the triglyceride phase comprises Sunflower seed oil. The sunflower seed oil may, for example, comprise from about 50-100% (e.g., about 85-95% by weight of the first phase).

The triglyceride phase may comprise additional ingredient(s) soluble in the triglycerides, including vitamins such as tocopheryl acetate, antioxidants, such as BHT and esters of ascorbic acid (e.g., ascorbic palmitate) and TDPA (e.g., dilauryl TDPA), preservatives such as parabens (methyl paraben) and phenoxyethanol, and botanical oils and essential oils. Additional oil soluble ingredients will collectively or individually comprise from about 0.001-20% by weight of the triglyceride phase. In one embodiment, the triglyceride phase will include from about 0.001-1% by weight tocopheryl acetate. In one embodiment, the triglyceride phase will include from about 0.001-5% by weight methyl paraben. In one embodiment, the triglyceride phase will include from about 0.001-0.5% by weight retinol. In one embodiment, the triglyceride phase will include from about 0.001-5% by weight phenoxyethanol. In one embodiment, the triglyceride phase will include from about 0.001-5% by weight BHT.

In some embodiments, the triglyceride phase will include less than 10% or less than 5% or less than 2.5% or less than 1% or less than 0.5%, or less than 0.25%, or less than 0.1% by weight glyceryl fatty acid monoesters and/or ethoxylated glyceryl fatty acid monoesters (e.g., PEG-glyceryl cocoate).

The compositions will also include a silicone oil phase. The silicone oil may comprise volatile or non-volatile silicone oils. The silicone oils may be linear or cyclic. Cyclic silicones including, for example, cyclomethicone pentamer, amodimethicones, dimethiconal may also be included in the silicone phase. In one embodiment, cyclic silicones may be utilized in the present invention, provided they do not render the silicone oil phase miscible with one or more of the other layers.

Linear silicones will generally have the structure:

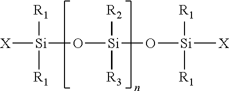

Where, “n” is an integer from 1-100,000 (or from 1-10,000 or from 10-5,000) such that the oil has a viscosity from about 0.65-500,000 cps (or from about 10-100,000 cps) at 25° C.; R₁-R₃ are selected from H, C₁₋₁₂ alkyl, alkenyl, alkynyl, aryl, alkyl-aryl, and aryl-alkyl, X₁, or -L-X₁; X is selected from H, —OH, C₁₋₁₂ alkyl, alkenyl, alkynyl, aryl, alkyl-aryl, and aryl-alkyl, a group -L-X₁, where L is a C₁₋₁₂ alkyl or a polyalkoxy group of the form —(CHR*CHR*O)p- or —(OCHR*CHR*)p- where R* is independently at each occurrence H or methyl, and “p” is an integer from 1-20; and X₁ is H, C₁₋₁₂ alkyl (e.g., methyl), —OH, —NH₂, —OH, or an epoxide of the form —CH(O)CH. In some embodiments, the silicone oil will comprise amodimethicone, dimethiconol, and/or dimethicone copolyol.

In one embodiment, the silicone oil phase will comprise from about 1-100% by weight, or from 2-100% or from 5-100% or from 25-100% or from 50-100% or from 75-100% or from 95-100% by weight of a linear dimethicone oil of the form:

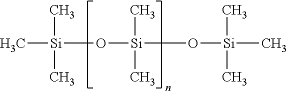

Where, “n” is an integer from 1-100,000 (or from 5-10,000 or from 10-5,000) such that the oil has a viscosity from about 0.65-500,000 cps (or from about 10-100,000 cps) at 25° C. (e.g., measured at a shear rate of 10 sec⁻¹).

The silicone phase may comprise additional ingredient soluble in silicone fluids. Additional silicone oil soluble ingredients will collectively or individually comprise from about 0.001-20% by weight of the silicone phase, provided their inclusion does not render the silicone phase miscible with the other phases.

The third phase comprises a polyalkylene glycol, such as polyethylene glycol (PEG) and/or polypropylene glycol (PPG). In some embodiment the third phase comprises polyethylene glycol (PEG). The polyalkylene glycol (e.g., PEG) may constitute from about 5-100% by weight of the third phase. In various embodiments, the polyalkylene glycol (e.g., PEG) may constitute from about 10-100%, about 20-100%, about 50-100%, or about 60-90% by weight of the third phase. The PEG may have, for example, a molecular weight (number average) from about 50-10,000 Daltons, or from about 100-1,000 Daltons, or from about 150-500 Daltons.

The polyalkylene glycol phase may comprise additional ingredients soluble or dispersible in the polyalkylene glycol, including alcohols (e.g., lower alcohols such as ethanol), water, sunscreens such as oxybenzone, preservatives such as parabens (methyl paraben) and phenoxyethanol, and pigments (e.g., FD&C lakes). Additional ingredients will collectively or individually comprise from about 0.001-20% by weight of the polyalkylene glycol phase. In one embodiment, the polyalkylene glycol phase will include from about 0.001-10% by weight organic sunscreen (e.g., oxybenzone). In one embodiment, the polyalkylene glycol phase will include from about 0.001-5% by weight methyl paraben. In one embodiment, the polyalkylene glycol phase will include from about 0.001-5% by weight phenoxyethanol.

In one embodiment, the polyalkylene glycol phase will include from about 1-45% or from 5-40% or from 10-35% or from 20-35% by weight ethanol. In one embodiment, the polyalkylene glycol phase will include from about 1-45% or from 5-40% or from 10-35% or from 20-35% by weight ethanol. In one embodiment, the polyalkylene glycol phase will include from about 0.1-20% or from 1-15% or from 1-5% by weight water. In some embodiments, the polyalkylene glycol phase will include less than 5% or less than 2.5% or less than 1% or less than 0.5%, or less than 0.25%, or less than 0.1% by weight water. In some embodiments, the polyalkylene glycol phase will be substantially anhydrous or anhydrous. In some embodiments, the polyalkylene glycol phase will include less than 10% or less than 5% or less than 2.5% or less than 1% or less than 0.5%, or less than 0.25%, or less than 0.1% by weight polyol, such as glycerin, sorbitol, and glycols such as hexylene glycol. In some embodiments, the polyalkylene glycol phase will include less than 10% or less than 5% or less than 2.5% or less than 1% or less than 0.5%, or less than 0.25%, or less than 0.1% by weight polyol, such as glycerin, sorbitol, and glycols such as hexylene glycol. In some embodiments, the polyalkylene glycol phase will include less than 10% or less than 5% or less than 2.5% or less than 1% or less than 0.5%, or less than 0.25%, or less than 0.1% by weight glyceryl fatty acid monoesters. In some embodiments, the polyalkylene glycol phase will include less than 10% or less than 5% or less than 2.5% or less than 1% or less than 0.5%, or less than 0.25%, or less than 0.1% by weight glyceryl fatty acid monoesters and/or ethoxylated glyceryl fatty acid monoesters (e.g., PEG-glyceryl cocoate). In some embodiments, the compositions are free of glyceryl fatty acid monoesters and/or ethoxylated glyceryl fatty acid monoesters.

The cosmetic and personal care products of the invention may comprise any conventional active and inactive ingredients, including colorants, fillers, powders, film forming polymers, gelling agents, waxes, thickeners, stabilizers, conditioners, solvents, emulsifiers, surfactants, humectants, emollients, sunscreens, pH adjusters, chelators, antioxidants, preservatives, fragrances, and the like.

Any of the phases may, independently from one another, comprise colorants, such as pigments and dyes. Colorants optionally may be present in the compositions, or in any of the phases, in amounts from 0.01-25% by weight.

As used herein, the term “pigment” embraces lakes (FD&C and D&C) and fillers or powders such as silica, talc, mica, calcium carbonate, etc. Exemplary inorganic pigments include, but are not limited to, inorganic oxides (e.g., metal oxides) and hydroxides such as magnesium oxide, magnesium hydroxide, calcium oxide, calcium hydroxides, aluminum oxide, aluminum hydroxide, iron oxides (e.g., α-Fe₂O₃, γ-Fe₂O₃, Fe₃O₄, FeO) and iron hydroxides including red iron oxide, yellow iron oxide and black iron oxide, titanium dioxide, titanium lower oxides, zirconium oxide, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. In one embodiment, the inorganic oxide particles may be selected from silica, alumina, zinc oxide, iron oxide, zinc oxide, and titanium dioxide particles, and mixtures thereof. In one embodiment, the pigments have a particle size from 5 nm to 500 microns, or from 10 nm to 100 microns, or from 100 nm to 30 microns or from about 0.75 to 20 microns. In some embodiments, the particle size (median) will be less than about 10 microns, less than about 5 microns or less than 1 micron.

Suitable lakes include, for example, FD&C lakes and D&C lakes. Lakes may include those based on barium, strontium, calcium or aluminum. Additional exemplary color additive lakes include, for example: D&C Red No. 19 (e.g., CI 45170, CI 73360 or CI 45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI#16035); FD&C Blue #1 (CI#42090); FD&C Yellow #5 (CI#19140); or any combinations thereof.

Additional suitable particulate colorants include carbon black, ultramarine blue, ferric blue, Prussian blue, manganese violet, talc, mica, sericite, calcium carbonate, fumed silica, and the like. Suitable pearling pigments include, without limitation, bismuth oxychloride, guanine, and titanated mica.

Pigments, if present, may be encapsulated in silica to impart soft-focus. The pigments may also be surface modified with hydrophobic and/or hydrophilic moieties, to adjust one or more characteristics of the colorant, such as dispersibility in the phase. For example, the pigments may be surface modified with alkylsilanes, including for example with Triethoxy Caprylylsilane or Triethoxy Caprylylsilane, as well as fluoro- and perfluoro-alkyl analogs thereof.

Suitable fillers may include talc, silica, alumina, zinc stearate, mica, kaolin, nylon (in particular orgasol) powder, polyethylene powder, polypropylene powder, acrylates powders, Teflon, starch, boron nitride, copolymer microspheres such as Expancel (Nobel Industrie), Polytrap (Dow Corning), and silicone resin microbeads (Tospearl from Toshiba). Other fillers that may be used in the compositions of the invention include inorganic powders such as chalk, fumed silica, fumed alumina, calcium oxide, calcium carbonate, magnesium oxide, magnesium carbonate, Fuller's earth, attapulgite, bentonite, muscovite, phlogopite, synthetic mica, lepidolite, hectorite, biotite, lithia mica, vermiculite, aluminum silicate, aluminum magnesium silicate, diatomaceous earth, starch, alkyl and/or trialkyl aryl ammonium smectites, chemically modified magnesium aluminum silicate, organically modified montmorillonite clay, hydrated aluminum silicate, hydrated silica, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicon dioxide; organic powder, cyclodextrin, methyl polymethacrylate powder, copolymer powder of styrene and acrylic acid, benzoguanamine resin powder, and poly(ethylene tetrafluoride) powder. Such additional powders and fillers optionally may be present in the compositions, or in any of the phases, in amounts from 0.01-25% by weight.

The compositions may include rheology modifiers such as gellants, thickeners, etc. The compositions may comprise a gelling agent, such an oil phase gelling agent. Gelling agents include, without limitation, the oil phase gellants dibutyl lauroyl glutamide and/or dibutyl ehtylhexyl glutamide. Gelling agents may also comprise, for example, one or more of a silicone resin, including Dimethicone/Vinyl Dimethicone crosspolymer, silicone T-resins, ETPEA, polyamides, cellulose ethers (e.g., methyl cellulose or ethyl cellulose) and the like. Thickeners such as acrylates copolymers, hydroxyalkyl cellulose, carboxymethylcellulose, carbomers, and vegetable gums such as xanthan gum may be included. Such rheology modifiers optionally may be present in the compositions, or in any of the phases, in amounts from 0.01-25% by weight.

The compositions may optionally include natural or synthetic film-forming polymers. Suitable polymeric film formers include polyolefins, silicone polymers (e.g., dimethicones, dimethiconols, amodimethicones, silicone resins, etc.), (meth)acrylates, alkyl (meth)acrylates, polyurethanes, fluoropolymers, silicone polyurethanes, and silicone acrylates such as acrylates/dimethicone copolymers. In some embodiments, it may be desirable to add a hydrophilic or water-soluble film former (e.g., cellulosics, polysaccharides, polyquaterniums (such as polyquaternium-37 (INCI), etc.) to the composition to improve spreading, stability, aesthetic look and feel, etc. Elastomers formed from ethylene, propylene, butylene, and/or styrene monomers may also be useful. Film forming polymers optionally may be present in the compositions, or in any of the phases, in amounts from 0.01-25% by weight.

Emollients (in addition to the triglyceride (e.g., vegetable oils such as sunflower oil), silicone, and PEG) may optionally be included in any of the phases. Suitable additional emollients include, without limitation, isopropyl myristate, petrolatum, volatile or non-volatile silicones oils (e.g., methicone, dimethicone), ester oils, mineral oils, hydrocarbon oils, and fatty acid esters. Such additional emollients optionally may be present in the compositions, or in any of the phases, in amounts from 0.01-25% by weight. Such additional emollients may be excluded from the scope of this invention to the extent that their presence will cause 2 or more of the phases to become miscible and/or stably miscible. In some embodiments, any or all of the layers may be free of additional emollients.

Humectants may optionally be included in any of the phases. Suitable humectants may include polyols (e.g., glycols), and may include without limitation glycerin, propylene glycol, ethoxydiglycol, butylene glycol, pentylene glycol, hexylene glycol, caprylyl glycol, and the like. These will typically be added in amount from about 0.001 to about 5% by weight. Humectants optionally may be present in the compositions, or in any of the phases, in amounts from 0.01-25% by weight. In some embodiments, the compositions comprise less than 10%, or less than 5%, or less than 2.5%, or less than 1% by weight of humectants (including polyols, such as glycerin and sorbitol, and glycols, such as hexylene glycol).

Waxes may optionally be included in any of the phases. Suitable waxes that may be used alone or in combination include, without limitation, natural waxes, mineral waxes, and synthetic waxes. Natural waxes are those of animal origin, including, without limitation, beeswax, spermaceti, lanolin, and shellac wax, and those of vegetable origin, including, without limitation, carnauba, candelilla, bayberry, and sugarcane wax. Mineral waxes contemplated to be useful include, without limitation, ozokerite, ceresin, montan, paraffin, microcrystalline, petroleum, and petrolatum waxes. Suitable synthetic waxes include, for example, polyethylene glycols such as PEG-18, PEG-20, PEG-32, PEG-75, PEG-90, PEG-100, and PEG-180 which are sold under the tradename CARBOWAX® (The Dow Chemical Company). Carbowax 1000 has a molecular weight range of 950 to 1,050 and a melting point of about 38° C., Carbowax 1450 has a molecular weight range of about 1,305 to 1,595 and a melting point of about 56° C., Carbowax 3350 has a molecular weight range of 3,015 to 3,685 and a melting point of about 56. ° C., and Carbowax 8000 has a molecular weight range of 7,000 to 9,000 and a melting point of about 61° C. Additional suitable synthetic waxes include Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX® Polyethylene (Baker Hughes Incorporated) with melting points ranging from 80° C. to 132° C. Commercially available ethylene-a-olefin copolymer waxes include those sold under the tradename PETROLITE® Copolymers (Baker Hughes Incorporated) with melting points ranging from 95° C. to 115° C. Waxes optionally may be present in the compositions, or in any of the phases, in amounts from 0.01-25% by weight.

A sunscreen (organic and/or inorganic) may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, bis-ethylhexloxphenol methoxyphenyl triazine, octyl triazone, titanium dioxide, zinc oxide, or any mixtures thereof. The sunscreen may be present from about 1% by weight to about 25% by weight of the total weight of the composition.

The compositions may further include an emulsifier, to the extent that the presence of an emulsifier or emulsifiers will not cause 2 or more of the phases to become miscible and/or stably miscible. The emulsifier may be ionic, zwitterionic, or nonionic. Suitable emulsifiers include those of the polyethoxylated type (e.g., polyoxyethylene ethers or esters), polydiorganosiloxane-polyoxyalkylene block copolymers (e.g., dimethicone copolyol), Steareth-20, Steareth-21, fatty alcohols (e.g., Cetearyl Alcohol), Polyoxethylene sorbitan fatty acid esters (i.e., polysorbates), and Hydrogenated Castor Oil, to name a few. Additional emulsifiers are provided in the INCI Ingredient Dictionary and Handbook 11th Edition 2006, the disclosure of which is hereby incorporated by reference. The amount of emulsifier will typically be from about 0.001 to about 10% by weight, based on the weight or the composition or the weight of an individual phase, but preferably will range from about 0.01 to about 5% by weight, and most preferably about 0.1 to about 1% by weight, based upon the total weight of the composition. In some embodiments, the phases may each, independently, be free of emulsifiers.

The compositions may comprise a cationic polymer. Cationic polymers include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. When present, the cationic polymer will typically comprise an amount of about 0.1% to about 15% by weight of the composition. In other embodiments the compositions may contain an amount of cationic (quaternium) ingredients that are anhydrous or have very low level of water, e.g., less than 1% by weight. Other suitable quaternium compounds include, without limitation, Polyquaternium-(INCI), Silicone Quaternium-18 (INCI), PEG-2 Dimeadowfoamamidoethylmonium Methosulfate and Hexylene Glycol (INCI), and Cetrimonium Chloride (INCI), to name a few. Such quaternium compounds, if present, will typically comprise from about 0.01% to about 10% by weight of the total composition or of a given phase, and more typically, from about 0.1% to about 1.5% by weight.

The compositions may also comprise monomer quaternary ammonium compounds such as, for example, alkyltrimethylammonium chlorides, dialkylmethyl-ammonium chlorides, alkyldimethylbenzylammonium chlorides, and alkylpyridinium chlorides. In one embodiment, the composition comprises at least one conditioning agent selected from the group consisting of polyquaterniums, cationic polymers, cationic surfactants, non-volatile dimethicone oils, dimethiconols, amodimethicones, ester oils, fatty alcohols, cationic gums and cellulosics, amidoamines, cetrimonium chloride, behentrimonium chloride, stearamidopropyl dimethylamine, polyesteramines, and cationically charge-modified polymers derived from guar gum, cellulose, proteins, polypeptides, chitosan, lanolin, starches and amino silicones. Such monomer quaternary ammonium compounds, if present, will typically comprise from about 0.01% to about 10% by weight of the total composition or of a given phase, and more typically, from about 0.1% to about 1.5% by weight.

The compositions may include a nonionic surfactant such as Laureth-23, Ceteth-10, Ceteth-20, IsoCeteth-20, Steareth-20, Oleth-10, Oleth-20, or alkyl polyglucose. The nonionic surfactant may be formed from a fatty alcohol, a fatty acid, or a glyceride with a C8 to C24 carbon chain. The compositions of the invention can further comprise proteins, peptides, and amino acids including hydrolyzed soy protein, lauryldimonium hydrolyzed soy protein (cationic Soya protein), wheat amino acids, corn, wheat, milk, or silk proteins, collagens, keratins, taurine and arginine hydrochloride, etc. Nonionic surfactants, if present, will typically comprise from about 0.01% to about 10% by weight of the total composition or of a given phase. In some embodiments, the compositions, or each individual phase, will comprises less than 5%, or less than 2.5%, or less than 1%, or less than 0.5%, or less than 0.25%, or less than 0.1% by weight surfactants, including ionic, nonionic, or zwitterionic surfactants. Surfactants may be utilized in the present invention, provided they do not render the silicone oil phase miscible and/or stably miscible with one or more of the other layers.

The cosmetic compositions of the invention may optionally include one or more agents that provide or enhance shine on a keratin fiber. Shine enhancing agents will typically have a refractive index greater than about 1.4, preferably greater than about 1.5 when measured as a film at 25° C. Suitable shine enhancing agents include without limitation, fatty esters, silicone, phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. When present, the shine-enhancing agent may be in an amount from about 0.1% to about 10% by weight, based on the total weight of the composition.

The compositions may also comprise a preservative or anti-microbial agent, for example, methylchloroisothiazolinone, methylisothiazolinone, methylparaben, propylparaben, phenoxyethanol, or caprylyl glycol. Such preservatives, if present, will typically comprise from about 0.0001% to about 10% by weight of the total composition or of a given phase, and more typically, from about 0.001% to about 1.5% by weight. In some embodiments, any one of the phases may, independent of the others, be free of preservatives.

The compositions of the invention may optionally include additional skin benefit agents such as antioxidants (e.g., BHT, ascorbic acid, sodium ascorbate, ascorbyl palmitate, beta-carotene, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinol esters such as acetates or palmitates), other anti-aging ingredients (e.g., collagen stimulators, collagenase inhibitors, elastase inhibitors), depigmenting agents (e.g., TDPA, hydroquinone, kojic acid), barrier function enhancing agents (e.g., ceramides, glycerides, cholesterol and its esters, alpha-hydroxy and omega-hydroxy fatty acids and esters thereof, etc.), exfoliating agents, estrogen synthetase stimulating compounds (e.g., caffeine and derivatives), compounds capable of inhibiting 5-alpha-reductase activity (e.g., linolenic acid, linoleic acid, finasteride, and mixtures thereof), perilla oil, and botanicals (e.g., tiliacora triandra). The compositions of the invention may also include a skin penetration enhancer; a skin plumper, such as palmitoyl oligopeptide, collagen, collagen and/or glycosaminoglycan (GAG) enhancing agents. These benefit agents will typically be present, if at all, in amounts, individually or in the aggregate, between about 0.001% and about 10% by weight of the composition or of the individual phases.

The cosmetic compositions and personal care products of the invention may be applied to the human integumentary system, including, skin, lips, nails, hair, and other keratinous surfaces. As used herein, the term “keratinous surface” refers to keratin-containing portions of the human integumentary system, which includes, but is not limited to, skin, lips, hair (including hair of the scalp, eyelashes, eyebrows, facial hair, and body hair such as hair of the arms, legs, etc.), and nails (toenails, fingernails, cuticles, etc.) of mammals, preferably humans. The compositions can be applied to any area of the skin, and preferably on the face, the neck, the hands, the feet, or other areas of the body, such as arms, legs, and back.

The cosmetic compositions may be in the form of any cosmetic or personal care product. For example, the cosmetic composition may be, without limitation, in the form of a color cosmetic, bath oil, soap, shampoo, conditioner, facial wash, body wash, sunscreen, insect repellant, self-tanner, moisturizer, etc.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

### Example 1

A three phase, three layer bath oil composition was prepared by separately preparing each of the First, Second and Third Phases according to Table 1. The three phases are added in any order to a clear container. In one embodiment, the First, Second and Third phases are added in amounts of about 25-40% by weight each. In one implementation, the First phase comprises about 31%, the second phase comprises about 34% and the Third phase comprises about 35%, thereby achieving approximately equal volumes of the three phases in the container.

TABLE 1

| | Wt. % |
|---|---|
| First Phase | |
| Sunflower seed oil | 80-100 |
| Methylparaben | <1 |
| Phenoxyethanol | <1 |
| Emollients | 0.1-15 |
| Vegetable oils | 0.1-10 |
| Tocopheryl acetate | <1 |
| Butylated hydroxytoluene | <1 |
| Second Phase | |
| Dimethicone oil | 100% |
| Third Phase | |
| Alcohol | 20-35 |
| Benzophenone-3 | .0001-1 |
| PEG 300 | 50-80 |
| Methylparaben | <1 |
| Phenoxyethanol | <1 |
| Pigments | .01-5 |

The container was subjected to repeated cycles of shaking followed by storage under quiescent conditions. There was not noticeable change in the volumes of each layer after repeated cycles of shaking and settling. In contrast, the identical composition was prepared by substituting the third phase with PEG-7 glyceryl cocoate. After repeated cycles of shaking followed by storage under quiescent conditions the bottom phase grew progressively smaller in volume. In some embodiments of the invention, the volume of each layer will not change by more than ±5% after 5, 10, 25, or 50 cycles of shaking followed by storage under quiescent conditions to allow the layers to fully separate.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A composition comprising a plurality of mutually immiscible liquid phases comprising:
(1) a first phase having triglyceride oil as the major component;
(2) a second phase having a silicone oil as the major component; and
(3) a third phase having polyethylene glycol (PEG) as the major component, wherein said third phase comprises from about 1-45% ethanol;
wherein said first, second and third phases are mutually immiscible and wherein the specific gravity of said second phase is greater than the first phase but less than the third phase, such that said first phase forms a top layer, said second phase forms a middle layer, and said third phase forms a bottom layer, the middle layer being in contact with the top and bottom layers,
wherein said layers are visually distinct from one another, and wherein said composition is substantially anhydrous.

2. The composition according to claim 1, wherein said first phase, second phase, and third phases form a single phase mixture upon shaking, but thereafter separate into distinct layers within 30 minutes on standing.

3. The composition according to claim 2, wherein the separation occurs within 10 minutes on standing.

4. The composition according to claim 1, wherein said first phase comprises sunflower oil in an amount from 50-100% by weight of said first phase

5. The composition according to claim 1, wherein said first phase comprises sunflower oil in an amount from 85-95% by weight of said first phase.

6. The composition according to claim 1, wherein said second phase comprises a linear silicone oil in an amount from 95-100% by weight of said second phase.

7. The composition according to claim 1, wherein said third phase comprises PEG in an amount from 50-100% by weight of said third phase.

8. The composition according to claim 1, wherein said third phase comprises PEG in an amount from 60-80% by weight of said third phase.

9. The composition according to claim 1, wherein said PEG has a number average molecular weight between about 100 and about 800 Daltons.

10. The composition according to claim 1, wherein said PEG has a number average molecular weight between about 200 and about 400 Daltons.

11. The composition according to claim 7, wherein said third phase comprises ethanol in an amount from 20-40% by weight of said third phase.

12. The composition according to claim 7, wherein said third phase comprises a colorant selected from pigments, lakes, and dyes.

13. The composition according to claim 7, wherein said composition is substantially anhydrous.

14. The composition according to claim 7, wherein said composition is substantially free of salts.

15. The composition according to claim 7, wherein said composition is substantially free of PEG-7 Glyceryl cocoate.

16. The composition according to claim 7, wherein said composition is substantially free of glycerin.

17. The composition according to claim 1, wherein:
(1) said first phase comprises from 80-100% by weight of sunflower oil, or another vegetable oil;
(2) said second phase comprises from 80-100% by weight silicone oil; and
(3) said third phase comprises (i) from 50-90% by weight of a liquid polyethylene glycol (PEG) having a number average molecular weight between about 100 and about 800 Daltons, and (ii) 10-40% by weight ethanol.

18. The composition of claim 17, wherein said first phase, second phase, and third phases form a single phase mixture upon shaking, but thereafter separate into distinct layers within 15 minutes on standing.

19. A packaged personal care product comprising:
(a) a container composed of a transparent or translucent material such that contents contained within said container are visible from outside the container, said container having a closable aperture for dispensing the contents thereof;
(b) a composition disposed within said container, the composition comprising:
(1) a first phase having triglyceride oil as the major component;
(2) a second phase having a silicone oil as the major component; and
(3) a third phase having polyethylene glycol (PEG) as the major component,
wherein said third phase comprises from about 1-45% ethanol;
wherein said first, second and third phases are mutually immiscible and wherein the specific gravity of said second phase is greater than the first phase but less than the third phase, such that, on standing, said first phase forms a top layer, said second phase forms a middle layer, and said third phase forms a bottom layer, the middle layer being in contact with the top and bottom layers, and wherein said layers are visually distinct from one another; wherein said first phase, second phase, and third phases form a single phase composition upon shaking, but separate back into distinct layers within 30 minutes on standing, and wherein said composition is substantially anhydrous.

20. The packaged personal care product according to claim 19 wherein said first phase, second phase, and third phase comprise approximately equal volumes.
